(19) United States
(12) Patent Application Publication
(10) Pub. No.: US 2015/0096568 A1
(43) Pub. Date: Apr. 9, 2015

(54) UPPER BODY SUPPORT DEVICE

(71) Applicant: Dario Grossberger, Camarillo, CA (US)

(72) Inventor: Dario Grossberger, Camarillo, CA (US)

(21) Appl. No.: 14/049,896

(22) Filed: Oct. 9, 2013

(51) Int. Cl. A61F 5/00

(52) U.S. Cl. CPC A61F 5/00
USPC 128/845

(57) ABSTRACT

An upper body support device. The upper body support device includes a chest support, wherein the chest support is configured to support the upper body of a user. The upper body support device also includes a leg brace, wherein the leg brace is configured to rest against the legs of the user. The upper body support device further includes a hinge connecting the chest support to the leg brace, wherein the hinge is configured to permit movement of the chest support relative to the leg brace.

### CROSS-REFERENCE TO RELATED APPLICATIONS

Not applicable.

### BACKGROUND OF THE INVENTION

Strawberry is generally grown in planting beds approximately one foot high, and the beds are accessed in one-foot wide aisles. Picking strawberry generally requires bending over, or stooping, to reach the strawberry fruit on the top and sides of the planting beds. Although workers shift positions somewhat, the stooped position is most efficient for strawberry harvest, and is thus generally maintained during hours of work. This position leads to fatigue and lower back pain and problems as it puts a lot of pressure on the vertebrae and muscles of the lower back.

The human back is prone to problems, so many inventions purport to provide it with support. Many inventions provide back support while seated and are thus not related to the present invention. Many inventions are similar to a wide belt, and are easy to put on, but provide relatively little support. In some inventions the belt is supplemented with additional straps which may go over the shoulder or straps which may go on the thighs or be integrated into apparel. These inventions are all soft, generally made of fabric or leather.

Other inventions use solid structures on the back (dorsal) side of the worker. These inventions generally replace the tension produced by muscles of the back with tension produced by the device. Although these inventions do provide support, they still require compression of the vertebrae of the back.

Other inventions use a spring support pressing against the leg to support the worker's upper body. The support is from the worker's ventral side, like the present invention, but the support is applied differently. In these inventions the force applied by the spring support itself supports the worker's upper body weight, and support is applied along the full arc of the back's motion as the worker stoops over. The inventions cited above use leg support derived from pressure upon the thigh.

Accordingly, there is a need in the art for a device which is designed to relieve stress on the lower back in the stooped position. In particular, there is a need for the device to support the upper body so the muscles need not contract and vertebrae need not be compressed in order to support the upper body while stooped. In addition, there is a need in the art for the device to support the worker's upper body from the front (ventral) side, not from the dorsal side. Moreover, there is a need in the art for the device to not apply support along the full arc of the back's motion. Also, there is a need in the art for a spring motion of the chest support only to raise the chest support so it does not interfere with work done by the worker while the worker is not stooped. Additionally, there is a need in the art for a device, which derives support from pressure against the shin, below the knee. This is essential because pressure from the upper body against the thigh will push the leg backwards, pushing the knee to a hyperextended position. When this pressure is instead applied against the shin it does not push to hyperextend the knee, so the pressure and the weight of the upper body are much more easily tolerated.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes an upper body support device. The upper body support device includes a chest support, wherein the chest support is configured to support the upper body of a user. The upper body support device also includes a leg brace, wherein the leg brace is configured to rest against the legs of the user. The upper body support device further includes a hinge connecting the chest support to the leg brace, wherein the hinge is configured to permit movement of the chest support relative to the leg brace.

Another example embodiment includes an upper body support device. The upper body support device includes a chest support, wherein the chest support is configured to support the upper body of a user. The upper body support device also includes a leg brace, wherein the leg brace is configured to rest against the legs of the user. The upper body support device further includes a hinge connecting the chest support to the leg brace, wherein the hinge is configured to permit movement of the chest support relative to the leg brace. The upper body support device additionally includes a spring assembly configured to retain the chest support in a predetermined position relative to the leg brace. The spring assembly includes a post mounted on the leg brace and a spring connected to the post. The spring assembly also includes a cable connected to the spring and the chest support and a roller configured to support the cable.

Another example embodiment includes an upper body support device. The upper body support device includes a chest support, wherein the chest support is configured to support the upper body of a user. The upper body support device also includes a leg brace, wherein the leg brace is configured to rest against the legs of the user. The upper body support device further includes a hinge connecting the chest support to the leg brace, wherein the hinge is configured to permit movement of the chest support relative to the leg brace. The upper body support device additionally includes a spring assembly configured to retain the chest support in a predetermined position relative to the leg brace. The spring assembly includes a post mounted on the leg brace and a spring connected to the post. The spring assembly also includes a cable connected to the spring and the chest support and a roller configured to support the cable. The upper body support device moreover includes a chest support stop configured to stop movement of the chest support relative to the leg brace at a predetermined angle. The upper body support device also includes a belt configured to attach the upper body support device to the waist of the user.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates a front view of an upper body support device;

FIG. 2 illustrates a front view of the upper body support device being used by a worker;

FIG. 3 illustrates a side view of the upper body support device;

FIG. 4A shows the position of the chest support when the worker is standing erect;

FIG. 4B shows the position of the chest support when the worker is partially stooped or bent over; and

FIG. 4C shows the position of the chest support when the worker is fully stooped and in the working position.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

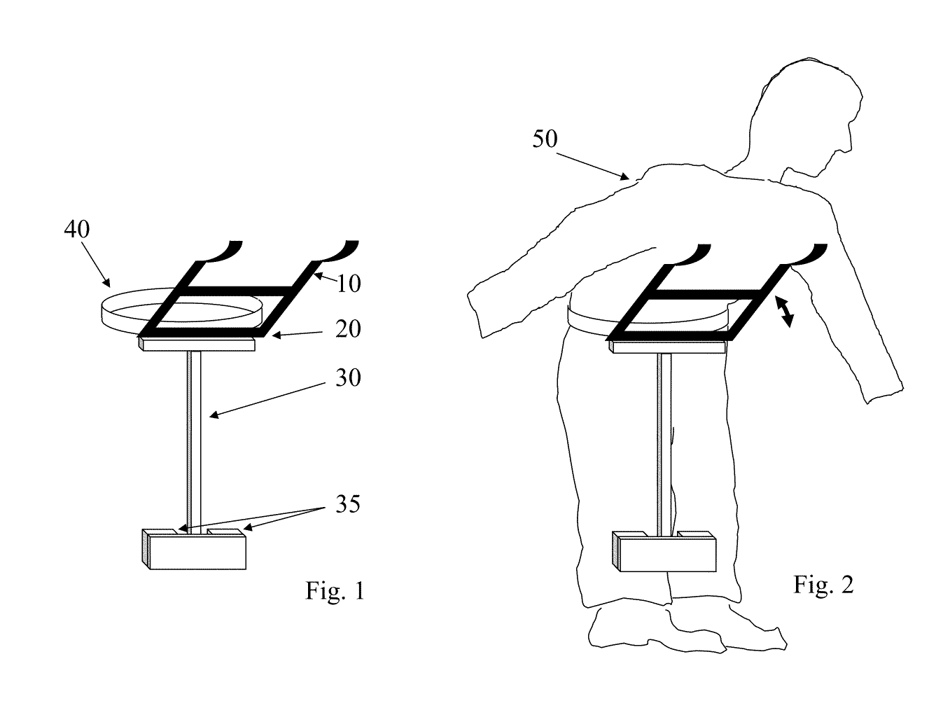

FIG. 1 illustrates a front view of an upper body support device. The chest support (10) is connected to a hinge (20). The other side of the hinge (20) is connected to the leg brace (30) which presses with cushions (35) against the shins, below the knees. The device is suspended on the waist of the worker with a belt (40). The stated application of the present invention is to strawberry picking. However, this can be used in other situations which require a stooped position, such as, but not limited to, harvest of other crops, such as celery or lettuce.

FIG. 2 illustrates a front view of the upper body support device being used by a worker. I.e., FIG. 2 is the same upper body support device shown in FIG. 1 is shown together with the worker (50) wearing the device.

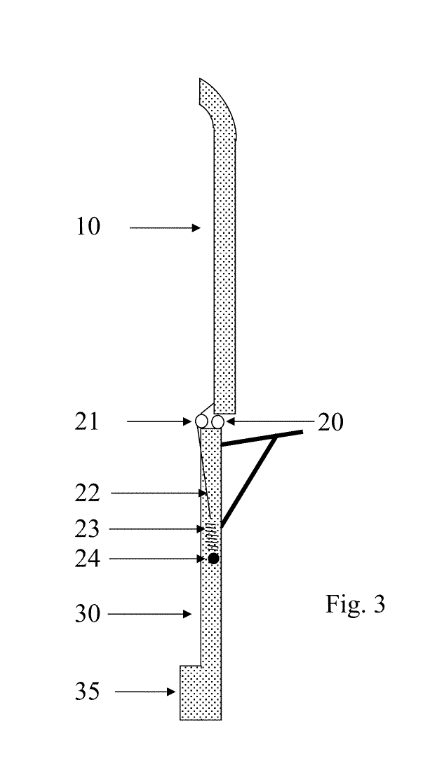

FIG. 3 illustrates a side view of the upper body support device. The chest support (10) is connected to one side of the hinge (20), and the other side of the hinge is connected to the leg brace (30). The chest support (10) is also connected to one end of a cable (22). That cable runs over a roller (21), and the other end of the cable is connected to one end of a spring (23). The other end of the spring is connected to the leg brace (30) by means of a post (24). Together these elements make the spring assembly (21, 22, 23, 24). The force exerted by the spring assembly returns the chest support (10) to the vertical position. The force applied by the spring is not intended to provide support to the upper body as the worker stoops, even though incidental force is applied to the upper body as an excess to the force needed only to raise the chest support itself.

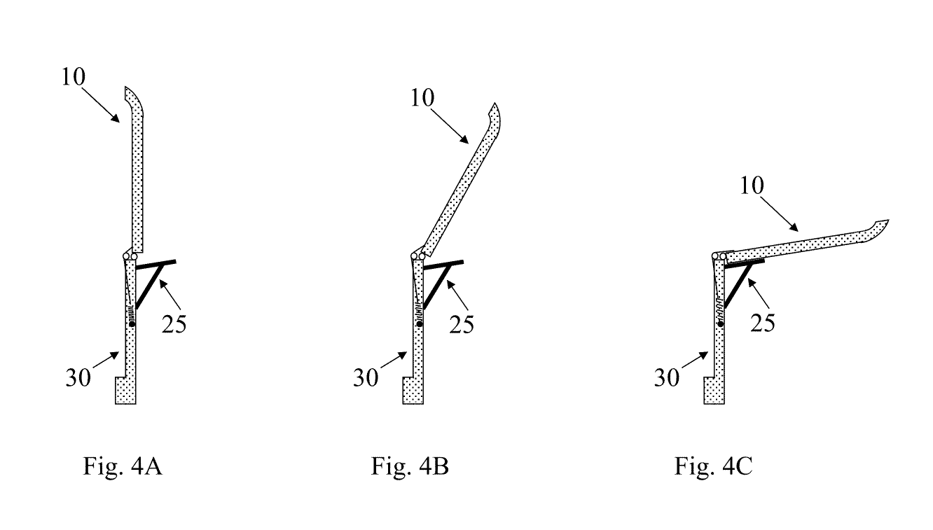

FIG. 4A shows the position of the chest support (10) when the worker is standing erect, with the chest support (10) maintained against the worker chest by the hinge (20) and spring assembly (21, 22, 23, 24). FIG. 4B shows the position of the chest support (10) when the worker is partially stooped or bent over. In this position the chest support (10) is maintained against the worker's chest by the hinge (20) and spring assembly but the force of the spring is insufficient to provide support of the worker's upper body. FIG. 4C shows the position of the chest support (10) when the worker is fully stooped and in the working position. In this position the rotation of the chest support (10) is stopped by the chest support stop (25), and the weight of the worker upper body is supported by the chest support (10).

One of skill in the art will appreciate that as the worker stands from the stooped position (moving from position in FIG. 4C to position in FIG. 4B to position in FIG. 4A) the upward rotation of the chest support (10) removes the chest support (10) from the work area of the worker. This improves worker comfort, mobility, and efficiency. In a different implementation the chest support (10) could be fixed in the position shown in FIG. 4C, so the chest support (10) would not rotate into the positions shown in FIGS. 4A and 4B. A fixed position such as in FIG. 4C is less desirable because the chest support (10) would interfere with any manual operations done while the worker is standing erect.

One of skill in the art will also appreciate that the amount of pressure produced on the chest support (10) could be raised. I.e., the upward force produced by the sprung hinge (20) on the chest support (10) could be increased so that that force itself is sufficient to support the weight of the worker upper body.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A upper body support device, the upper body support device comprising:
a chest support, wherein the chest support is configured to support the upper body of a user;
a leg brace, wherein the leg brace is configured to rest against the legs of the user; and
a hinge connecting the chest support to the leg brace, wherein the hinge is configured to permit movement of the chest support relative to the leg brace.

2. The upper body support device of claim 1 further comprising a cushion on the leg brace.

3. The upper body support device of claim 1 further comprising a spring assembly, wherein the spring assembly is configured to retain the chest support in a predetermined position relative to the leg brace.

4. The upper body support device of claim 3, wherein the spring assembly includes a post connected to a spring.

5. The upper body support device of claim 4, wherein the post is mounted to the leg brace.

6. The upper body support device of claim 3, wherein the spring assembly includes a cable connected to the upper body support.

7. The upper body support device of claim 6, wherein the cable passes over a roller.

8. A upper body support device, the upper body support device comprising:
a chest support, wherein the chest support is configured to support the upper body of a user;
a leg brace, wherein the leg brace is configured to rest against the legs of the user; and
a hinge connecting the chest support to the leg brace, wherein the hinge is configured to permit movement of the chest support relative to the leg brace; and
a spring assembly configured to retain the chest support in a predetermined position relative to the leg brace, wherein the spring assembly includes:
a post mounted on the leg brace;
a spring connected to the post;
a cable connected to the spring and the chest support; and
a roller configured to support the cable.

9. The upper body support device of claim 8 further comprising a chest support stop configured to stop movement of the chest support relative to the leg brace at a predetermined angle.

10. The upper body support device of claim 8 further comprising a belt configured to attach the upper body support device to the waist of the user.

11. A upper body support device for, the upper body support device comprising:
a chest support, wherein the chest support is configured to support the upper body of a user;
a leg brace, wherein the leg brace is configured to rest against the legs of the user; and
a hinge connecting the chest support to the leg brace, wherein the hinge is configured to permit movement of the chest support relative to the leg brace;
a spring assembly configured to retain the chest support in a predetermined position relative to the leg brace, wherein the spring assembly includes:
a post mounted on the leg brace;
a spring connected to the post;
a cable connected to the spring and the chest support; and
a roller configured to support the cable;
a chest support stop configured to stop movement of the chest support relative to the leg brace at a predetermined angle; and
a belt configured to attach the upper body support device to the waist of the user.
